(19) United States
(12) Patent Application Publication
(10) Pub. No.: US 2005/0240177 A1
(43) Pub. Date: Oct. 27, 2005

(54) Electrosurgical forceps

(75) Inventors: Nunilon F. Tabermejo JR., Louisville, KY (US); Jack A. Stroski, Georgetown, IN (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Packaging Service Corporation of Kentucky, Louisville, KY (US)

(21) Appl. No.: 11/114,353

(22) Filed: Apr. 26, 2005

Related U.S. Application Data
(60) Provisional application No. 60/565,294, filed on Apr. 26, 2004.

(51) Int. Cl. A61B 18/14

(52) U.S. Cl. 606/42; 606/51; 606/52

(57) ABSTRACT

The invention includes an electromechanical hand-switching forceps arrangement that includes a switch in the form of an insulated pin and an insulated cylinder block to receive the pin midway along the respective tines. The insulated pin and insulated cylinder block contain respective contacts. Except for the contacts themselves, the switch is fully enclosed and fully insulated so that when it is activated, there is no leakage of high frequency electrosurgical current in the surrounding area. The pin is normally disposed just within the opening in the cylinder block such that they are not in contact with the closing of the tips of the tines.

### FIELD OF THE INVENTION

The present invention relates to forceps for use in deep body cavity surgery, and more particularly to a forceps for use in division of tissues of haemostasis in such surgeries.

### BACKGROUND OF THE INVENTION

It is a common practice in some surgical techniques to utilize electrical current for the cauterization or electro-coagulation of small blood vessels and the like. While foot switches operated by the surgeon have been used to operate such devices, the shortcomings of this arrangement have led to the development of alternate devices that provide safer and more sophisticated control of the cauterization instrument. For example, in the 1960s, relay operated electrosurgical forceps were developed, such as disclosed in U.S. Pat. No. 3,100,489 to Bagley. In the forceps of Bagley, closure of the forceps itself closes a switching means to provide radio frequency electrical energy to a site to be treated.

In this type of arrangement, typically, the contacts of the switch on the forceps are exposed to permit engagement thereof when the forceps are closed. Inasmuch as the potentials on these exposed contacts can be substantial, however, there is a certain degree of danger associated therewith. The danger of inadvertently burning unintended tissue is magnified in deep body cavity type surgery, particularly where the surgeon is operating through a relatively small incision. Further, in surgeries where excessive moisture is present, such fluid can cause inadvertent electrical contact and activation of electrical energy.

In an attempt to remedy these shortcomings, hand-switch operated electrosurgical forceps were developed. Such an arrangement is disclosed, for example in U.S. Pat. No. 4,041,952 to Morrison, Jr. In hand-switch forceps, a switching member is disposed on only one tine of the forceps, and is operated independently of the closing of the forceps. This type of arrangement, however, likewise has its shortcomings. Most notably, the operation of the switching member is sometimes cumbersome. Moreover, it requires the extra step of actuating the switch, rather than the automatic operation upon closure of the forceps.

As a result, it is desirable to provide an electrosurgical forceps arrangement that overcomes the shortcomings and maintains the advantages associated with the various arrangements known in the art.

### BRIEF SUMMARY OF THE INVENTION

The invention provides an electromechanical forceps arrangement that includes a switch in the form of an insulated pin and an insulated cylinder block to receive the pin midway along the respective tines. The insulated pin and insulated cylinder block contain respective contacts. Except for the contacts themselves, the switch is fully enclosed and fully insulated so that when it is activated, there is no leakage of high frequency electrosurgical current in the surrounding area. The distal end of the pin is normally disposed within the opening in the cylinder block such that they are not in contact with the closing of the tips of the tines. The gap between the contacts is sized such that there is no uninsulated part of the switch that is exposed when the instrument tips are open. As a result, the electrosurgical hand-switching device allows the instrument to be in close proximity to tissue or with the pocket of the surgical site without the danger of burning the surrounding areas around the switch with unintended spark or contact. When the switch contacts are touching each other, however, it activates the flow of high frequency current from an energy source, such as an electrosurgical generator to the surgical site.

In use, the surgeon brings the tines of the forceps together to touch the tips to the desired surgical site. Additional force is required, however, to bring the midsections of the tines further toward each other in order to touch the switch contacts. Thus, once in such proper position, the surgeon squeezes the central portions of the tines slightly further to cause the uninsulated contact at the distal tip of the pin to engage the second contact at the uninsulated base of the cylinder block to activate electrical current to the tips of the tines.

In a monopolar application, the instrument comprises an active electrode (+), while the dispersive electrode is remotely attached to the patient body, which is the ground (−). Conversely, in a bipolar application, both poles are contained within the instrument, either tip of the respective tine being the active electrode or the ground. In both monopolar and bipolar application, the switch is attached to the forceps, which is connected to the electrosurgical generator by a cable. Accordingly, the surgeon may remotely and instantaneously activate the electrosurgical generator by closing or touching the switch contacts together.

The switch contacts can be made from any conductive and biocompatible material. Inasmuch as the device is preferably reusable, the switch insulation or shielding can be any biocompatible material that withstands high frequency electrosurgical voltage at elevated levels, and can be sterilized in any manner of hospital sterilization process.

These and other objects and advantages of the invention will be apparent to those skilled in the art upon reading the following detailed description and upon reference to the drawings.

### BRIEF DESCRIPTION OF THE DRAWINGS

FIG. 1 is a side view of a hand-switching forceps assembly constructed in accordance with teachings of the art.

FIG. 2 is a plan view of the hand-switching forceps of FIG. 1, a sleeve being partially broken away.

FIG. 3 is an enlarged, partial cross-sectional view of the contact switch of the hand-switching forceps of FIG. 2 in an open position.

FIG. 4 is an enlarged, partial cross-sectional view of the contact switch of the hand-switching forceps of FIG. 2 in a closed position.

FIG. 5 is an exploded view of the contact switch of FIGS. 3-4.

### DETAILED DESCRIPTION OF THE INVENTION

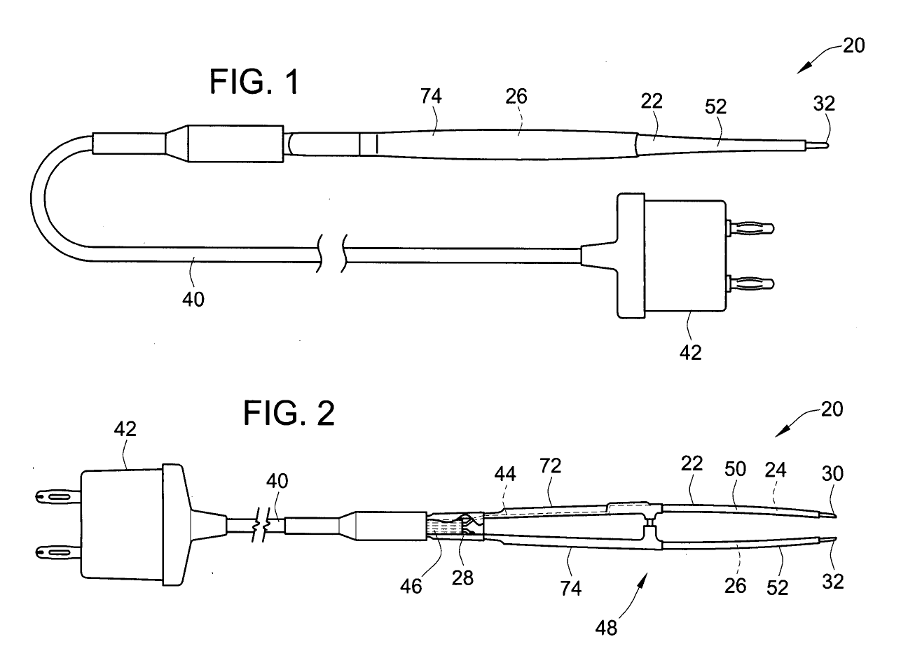

Turning now to the drawings, there is shown in FIGS. 1 and 2, there is a hand-switching forceps assembly 20 constructed in accordance with teachings of the invention. The forceps assembly 20 includes a surgical forceps 22 having a first tine 24 and a second tine 26 coupled at their proximal ends at a sleeve 28. The tines 24, 26 may be so coupled by any appropriate means. The tines 24, 26 are typically formed of stainless steel, nickel-plated brass, titanium, or any composite material with a conductive core. The distal ends of the tines 24, 26 present tips 30, 32. When the tines 24, 26 are advanced toward one another, the tips 30, 32 touch, allowing the surgeon to grasp tissue or another item such as a suture.

In use, as the tips 30, 32 of the forceps 22 are closed around tissue (not shown), electrosurgical current is passed to through the tines 24, 26 to the tips 30, 32 such that it is applied to the grasped tissue to cauterize or electro-coagulate small blood vessels at a surgical site. Power is typically supplied to the surgical forceps 22 from a power source (not shown) by way of a cable 40 that may be coupled to the power source by ajack or plug or other appropriate fitting arrangement 42. While the illustrated embodiment includes a two-prong jack 42, the fitting arrangement 42 could alternately include a three-prong jack, for example, as is known in the industry. The illustrated embodiment comprises a monopolar cable 40 comprising two wires 44, 46. It will be appreciated, however that a bipolar arrangement may alternately be utilized in keeping with the invention. The wires 44, 46 are electrically coupled to the respective tines 24, 26 such that when the tips 30, 32 are closed around the tissue and upon closing of a switching assembly (indicated generally as 48), power is supplied to one or both of the tips 30, 32. The arrangement for electrically coupling the wires 44, 46 to the tines 24, 26 will be explained in greater detail below.

As may best be seen in FIG. 2, the forceps 22 includes primary insulative covers 50, 52 which substantially cover the length of the tines 24, 26 with the respective tips 30, 32 being left uncovered. The insulative covers 50, 52 may be formed of any appropriate material. Typically, the insulative covers 50, 52 are formed of plastic, rubber or the like. It will be appreciated that such insulation must be biocompatible and withstand the high frequency electrosurgical voltage at elevated levels. Further, if the forceps is to be autoclavible, the insulation must be able to withstand such sterilization processes. While any appropriate method may be utilized to form the covers 50, 52, dip-coating and shrink-wrapping have been found to be particularly useful processes by which the insulative covers 50, 52 may be applied to the tines 24, 26.

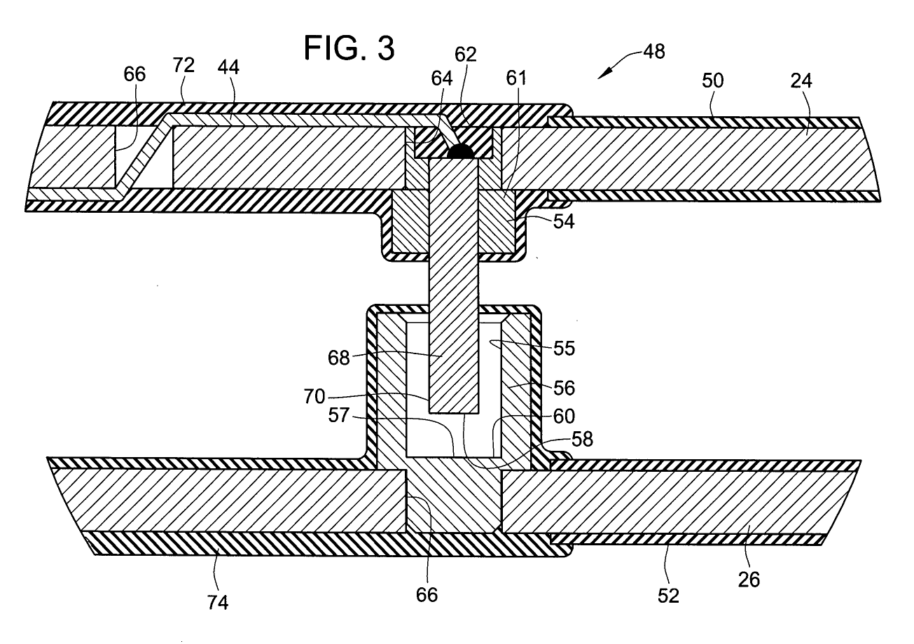

In accordance with the invention, the switching assembly 48 is fully enclosed and insulated such that electrosurgical current may be accurately, selectively provided to the surgical site, substantially eliminating undesirable arcing that may result in an unintended spark or flow of power. More specifically, and as shown in more detail in FIG. 3, the switch assembly 48 comprises an insulated pin 54 and an insulated cylinder block 56 that contain respective contacts 58, 60. In this way, the connection to supply current to the tips 30, 32 of the tines 24, 26 is not made until the contacts 58, 60 touch to make the electrical connection.

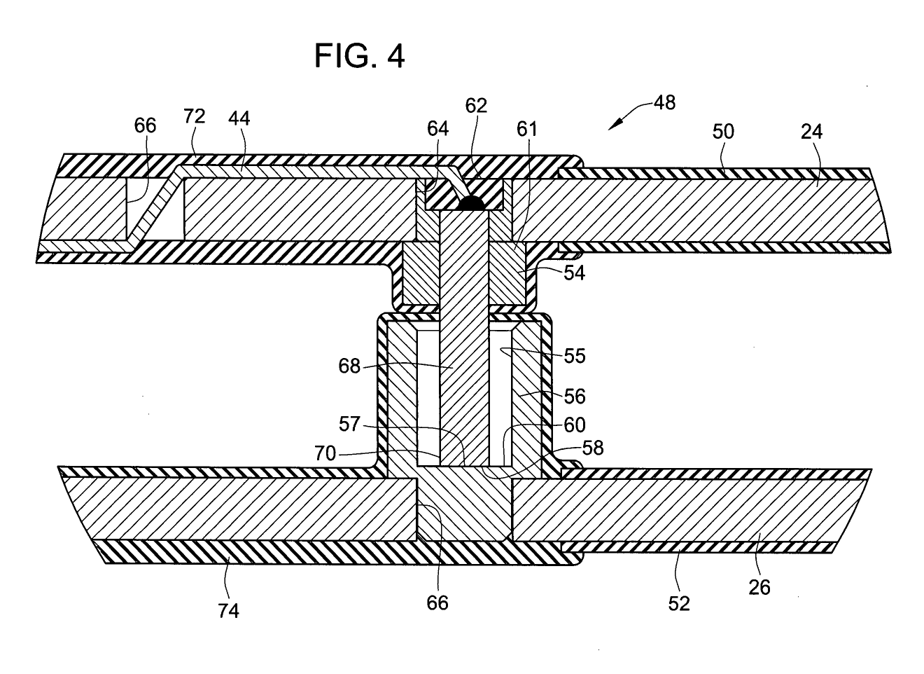

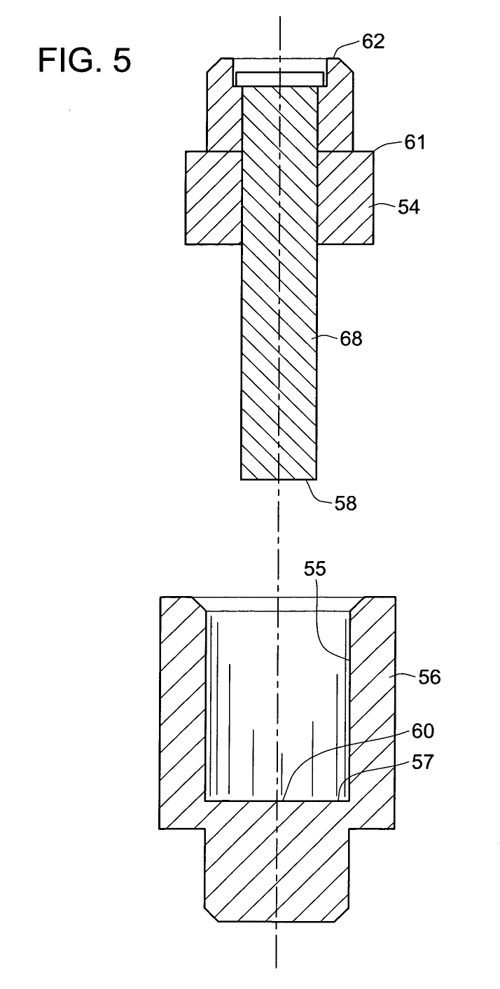

In a currently preferred embodiment of the invention, the pin 54 comprises an enlarged head 61 and a probe portion 68. The proximal end 62 of the pin 54, here, a portion of the head 61, is preferably received in a first bore 64 in one of the tines 24, while the cylinder block 56 is received in a bore 66 in the other tine 26. In this way, the pin 54 and cylinder block 56 extend toward one another between the tines 24, 26, the probe portion 68 of the pin 54 being disposed within the channel 55 of the cylinder block 56 with the contact 58 at the distal end 70 of the pin 54 being spaced slightly apart from the contact 60 at the base 57 of the cylinder block 56 in the free position (see FIG. 3). Preferably, in use, the tips 30, 32 of the tines 24, 26 may be brought together to grasp the target tissue, and the midsections of the forceps 22 may be squeezed slightly more to bring the tines 24, 26 closer together to cause the contacts 58, 60 to touch and close the switch 48 (see FIG. 4).

The switch 48 is connected to the power supply by the wires 44, 46, which are preferably jacketed. As shown in FIG. 2, in the illustrated monopolar forceps illustrated, one of the wires 46 is connected directly to the forceps 22 at the tine 26 from which the cylinder block 56 extends. The second wire 44 preferably extends along the inside of the tine 24 then through a second bore 66 in the tine 24 to the outside of the tine 24. The wire 44 then connects to the proximal end 62 of the pin 54 by soldering or the like, as shown in FIG. 3. In this way, as the contacts 58, 60 are brought together, as shown in FIG. 4, connection is made to supply current to the tips 30, 32 of the tines 24, 26.

According to another important feature of the invention, the switching assembly 48, and, more particularly, the pin 54 and cylinder block 56 are insulated to prevent inadvertent closing of the switch 48 and supply of current to the tips 30, 32. In the currently preferred embodiment, sleeves 72, 74 of insulation are disposed about the wire 44 and the tines 24, 26, with the insulative sleeves 72, 74 substantially covering all of pin 54 and cylinder block 56 except the probe portion 68 of the pin 54 and the channel 55 of the cylinder block 56. While the insulative sleeves 72, 74 may be of any appropriate material, in the currently preferred embodiment, a polymeric material such as Kynar is utilized. As with the primary insulative sleeves, the secondary insulative sleeves 72, 74 must by formed of a biocompatible material that withstands high frequency electrosurgical voltage at elevated levels, as well as the rigors of autoclaving. Further, while the insulative sleeves 72, 74 may be applied in any appropriate manner, in the currently preferred embodiment, the insulative sleeves 72, 74 are overwraps that are then heat shrunk to the tines 24, 26. It will be appreciated, however, that alternate materials, arrangements or assembly methods may be utilized within the scope of the present invention.

Preferably, the forceps 22 may be repeatedly autoclaved or otherwise sterilized for repeated use. Alternately, the forceps assembly 20 may be utilized and discarded thereafter.

While this invention has been described with an emphasis upon preferred embodiments, variations of the preferred embodiments can be used, and it is intended that the invention can be practiced otherwise than as specifically described herein. Accordingly, this invention includes all modifications encompassed within the spirit and scope of the invention as defined by the following claims.

All of the references cited herein, including patents, patent applications, and publications, are hereby incorporated in their entireties by reference.

1. A hand-switching electrosurgical forceps for coupling to an energy source, the forceps comprising:
a first tine having a distal end and a proximal end,
a second tine having a distal end and a proximal end, the first and second tines coupled together at their proximal ends, said distal ends being substantially adjacently disposed but and spaced apart, the tines being resilient such that a force applied to the tines moves the distal ends of the tines toward one another,
a switch coupled to the energy source and comprising a pin secured to the first tine and a cylinder block secured to the second tine, the pin having a distal end comprising a contact, the cylinder block comprising an internal bore and an internal contact within said internal bore, the distal end of the pin being disposed within the cylinder block and the pin and cylinder block being disposed for relative axial movement as the force is applied to the tines,
a first insulative cover disposed about the first tine such that the distal end of the tine is exposed and at least the pin contact is exposed, and
a second insulative cover disposed about the second tine such that the distal end of the tine is exposed and at least the internal contact is exposed, the first and second insulative covers being substantially impermeable to fluids,
said switch being manually closable by axial movement of the pin within the cylinder block to cause the pin contact and the internal contact to engage, the switch causing energy to be supplied to the distal ends of the tines when the contacts are engaged.

2. The forceps of claim 1 wherein the forceps is monopolar.

3. The forceps of claim 1 wherein the forceps is bipolar.

4. The forceps of claim 1 wherein the pin comprises a head portion and a distally disposed probe, and the first tine comprises a bore, at least a portion of the head being disposed within the bore of the first tine, and first insulative covering being disposed about at least a portion of the head, at least the distal end of the probe being without said first insulative cover.

5. The forceps of claim 1 wherein the second tine comprises a bore, at least a portion of the cylinder block being disposed within the bore of the second tine.

6. The forceps of claim 1 wherein the cylinder block comprises a base, the base being disposed within the cylinder block at an end of the internal bore, the internal contact being disposed substantially adjacent said base.

7. The forceps of claim 1 further comprising at least a first and a second lead wire, the first lead wire being electrically coupled to the energy source and the distal end of the first tine, the second lead wire being electrically coupled to the energy source and the distal end of the second tine.

8. The forceps of claim 7 wherein the first lead wire is coupled to and terminates at the pin.

9. The forceps of claim 8 wherein the first tine comprises an inside surface disposed toward the second tine, and an outside surface disposed away from the second tine, and a bore extending between the inside and outside surfaces, the first lead wire being disposed along at least a portion of the inside surface, extending through the bore between the inside and outside surfaces, and extending along at least a portion of the outside surface.

10. The forceps of claim 7 wherein the second lead wire is coupled to and terminates at the second tine.

11. The forceps of claim 1 wherein a first force is required to cause the distal ends of the tines to touch, and a second force is required to engage the contacts, the second force being greater than the first force.

12. The forceps of claim 1 wherein the insulative covers comprise a primary insulative cover over the tines and a secondary insulative cover over the switch.

13. The forceps of claim 1 further comprising a cable and a fitting for coupling the tines to the power source.

14. The forceps of claim 13 wherein the fitting comprises a two-prong jack.

15. The forceps of claim 13 wherein the fitting comprises a three-prong jack.

16. The forceps of claim 1 wherein substantially only the distal end of the first tine and the pin contact are the only portions of the first tine that are not covered by the first insulative cover.

17. The forceps of claim 1 wherein substantially only the distal end of the second tine and the internal contact are the only portions of the second tine that are not covered by the second insulative cover.
